(19) United States
(12) Patent Application Publication
(10) Pub. No.: US 2012/0186596 A1
(43) Pub. Date: Jul. 26, 2012

(54) Compositions And Methods For Permanent Straightening Of Hair

(76) Inventors: Jean Harry Xavier, Holbrook, NY (US); Geoffrey Hawkins, Yardley, PA (US); Thomas Mammone, Farmingdale, NY (US); Lavinia C. Popescu, Jackson Heights, NY (US)

(21) Appl. No.: 13/013,482

(22) Filed: Jan. 25, 2011

(51) Int. Cl. A61K 8/66; A45D 7/06; A61Q 5/04

(52) U.S. Cl. 132/206; 424/70.2; 424/70.5

(57) ABSTRACT

A hair straightening topical composition comprising transglutaminase and polylysine, which act to form a surface barrier film and moisture shield around human hair. The transglutaminase also contributes to hair straightening. The composition also comprises one or more additional hair straightening agents, other than TGase, that are capable of affecting secondary, tertiary and quaternary protein structures of human hair, preferably sodium metabisulfite and/or tourmaline. The invention includes compositions that may be washed out of the hair after straightening has occurred, and compositions that are intended to remain in the hair for additional or extended benefits. The invention includes methods of using a topical composition that is capable of affecting secondary, tertiary and quaternary protein structures of human hair. Testing indicates that the hair straightening is long term, occurs significantly faster than known commercial hair straightening compositions, and there is significantly less damage to hair compared to known heat and chemical treatments.

### FIELD OF THE INVENTION

The invention is in the field of hair straightening. More particularly, it is in the field of improved compositions and methods for permanent straightening of human hair.

### BACKGROUND OF THE INVENTION

Various methods of straightening hair are known, each having it's own advantages and disadvantages to a more or lesser degree. Hair straightening technologies include those that alter the primary protein structure of hair, those that alter secondary or tertiary protein structure, and those that hold hair in a straightened configuration against the hair's natural tendency to curl.

### Human Hair

U.S. Pat. No. 5,395,490 is herein incorporated by reference, in its entirety. Several figures in U.S. Pat. No. 5,395,490 diagram the structure of human hair fibers, the protein components of hair, and energy levels of the disulfide bond. A fiber of human hair comprises three main morphological components: the cuticle, the cortex, and the cell membrane complex, which itself is comprised of a protein matrix of keratin peptide chains.

The natural shape and structural integrity of human hair depends, in part, on the orientation of disulfide bonds (cysteine-cysteine bonds). In human hair, disulfide bonds that link one part of a peptide chain with another part of the same chain contribute to tertiary protein structure, while disulfide bonds that link two different peptide chains contribute to quaternary structure. Furthermore, disulfide bonds are known to protect secondary protein structures in the immediate vicinity of the bond. They may do this by shielding hydrogen bonds.

Thus, it is generally thought that alteration of the disulfide bonds is necessary and/or useful to effect long term changes in the shape of hair. Hair shaping treatments that do not rearrange the disulfide bonds result in changes in hair shape that last a relatively short time. For example, the use of heat to style hair may create temporary straightening of the hair. However, the styled hair will return to its natural shape after a short time, as a result of exposure to moisture in the air or washing. The use of heat and moisture to straighten hair may break and reconfigure hydrogen bonds in the hair, but the disulfide bonds are not substantially affected. It is thought that hydrogen bonds, by themselves, are insufficient to hold the shape of hair for a significant time, because the stronger disulfide bonds eventually force the hair to reassume its original shape. Thus, a permanent straightening of the hair is thought to involve the cleaving and reforming of a substantial number of disulfide bonds. Various chemical treatments for doing this are known.

### Chemical Hair Straightening Treatments

Hair straightening by treating the hair with chemical agents is well known. Depending on the straightening agent used, damage to the protein structure may be controlled to a more or less degree. That is, various types of protein structures of the treated hair may be broken down, or only a select type of protein structure. For example, hair straightening products that alter primary structure, do so by weakening and/or breaking the internal chemical bonds of hair protein amino acids. Regardless of where the protein structure is altered, effective straightening treatments cause natural curls to loosen and straighten. While some straightening agents may be more effective and/or efficient than others, the trade off is usually in the damage done to the hair and scalp, and the need for adjunct treatments to limit that damage. On the other hand, treatments which may be somewhat less damaging to the hair and scalp, may require a longer time to operate, or the application of significantly more product, or multiple applications to achieve a desired result.

Among known hair straightening products that alter primary structure we may name products comprising sodium hydroxide, potassium hydroxide, lithium hydroxide, and guanidine hydroxide. It is generally acknowledged that repeated use of hydroxide hair straightening products can be very damaging to hair. Among hair straightening products that disrupt tertiary, and perhaps quaternary structure, we can name ammonium thioglycolate, ammonium sulfite, ammonium bisulfite, sodium metabisulfite (Na₂S₂O₅), and cysteine. These sulfur-containing agents are more targeted in the damage that they inflict on hair proteins. These agents act primarily by selectively weakening or cleaving the disulfide bonds in cystine, instead of disrupting the entire protein. First, the sulfur-containing agent is used to reduce the disulfide bonds, along with the application of mechanical stress. Next, new disulfide bonds are allowed to form, in a new arrangement, thus giving the hair a new shape. An oxidizing agent may be used to help constitute the new disulfide bonds. In the art of hair straightening, repeated use of ammonium thioglycolate or cysteine is considered significantly damaging to hair, while ammonium sulfite and ammonium bisulfite also cause damage.

Hair straightening products based on formaldehyde are also known, and have come under some scrutiny by health authorities in recent months. In general, known chemical treatments are considered harsh and damaging to human hair and skin. The damage done to hair is measured as a loss of cystine content (fewer S—S bonds indicating a loss of protein structure), a decrease in water contact angle (loss of hydrophobicity), an increase in microscopic damage to cuticle (swelling and lifting), a decrease in mechanical break strength. Some visible manifestations of the negative effects of chemical hair styling include dry, brittle or limp hair, and a loss of shine and/or color.

### Transglutaminase

Transglutaminases are a family of enzymes that are found naturally throughout the body, including the hair. In the prior art, transglutaminase (hereinafter, TGase) catalyzes the posttranslational modification of proteins by transamidation of available glutamine residues. Transglutaminases are calcium dependent enzymes with the ability to covalently bond protein bound glutamine and protein bound lysine. A major result of this activity is glutamyl-lysine cross-links in proteins. Glutamine is readily available in hair, while lysine is present to lesser degree. Used topically, TGase may contribute to the overall protein structure of hair.

Commonly owned, co-pending application, US2009-0126754, discloses the use of topically applied transglutaminase (without any free lysine) to retain curl in curled hair. However, it was also reported that when Activa™ TG-TI (a commercially available transglutaminase blend) was present at concentrations of 2%, 5% and 10%, the product caused curled hair to droop, within 30 minutes of application, by as much 25%, 33% and 16% respectively. Despite this, curl was not eliminated completely, and the hair was not straightened to a sufficient degree, to make a commercially viable hair straightening product based on TGase alone.

A number of other topical uses for transglutaminases have been proposed. JP 2719166 discloses compositions containing transglutaminase and a polyhydric alcohol, said to be useful in treatment of damaged hair by increasing the moisture retention of the hair. JP 3-083908 suggests the use of transglutaminase in combination with polyethylene glycol or other water soluble materials to treat chapped skin. It has also been suggested for use in binding active components to skin, hair or nails (U.S. Pat. No. 5,490,980). WO01/21145 teaches the use of transglutaminase to improve the color-fastness of hair dyes. WO01/21139 suggests a combination of transglutaminase and an active substance having substrate activity for transglutaminase, for use in restructuring damaged keratin fibers. U.S. Pat. No. 5,525,336 discloses the combination of corneocyte proteins and transglutaminase for application to skin, hair or nails to form a protective layer.

### Tourmaline

Hair straightening products that alter secondary or tertiary structure include those that comprise tourmaline. Tourmaline is an acentric rhombohedral borosilicate characterized by six-membered tetrahedral rings. It is a semi-precious stone, and a crystal silicate compounded with varying amount of elements such as aluminum, iron, magnesium, sodium, lithium, or potassium. The beneficial effects and advantages of heat activated tourmaline on hair proteins has been discussed at length in commonly owned applications WO2010/096598, WO2010/096605, and WO2010/096610, herein incorporated by reference, in their entirety.

### SUMMARY OF THE INVENTION

The present invention is a hair straightening topical composition comprising transglutaminase and polylysine, which act to form a surface barrier film and moisture shield around human hair. The transglutaminase also contributes to hair straightening.

The composition also comprises one or more additional hair straightening agents, other than TGase, that are capable of affecting secondary, tertiary and quaternary protein structures of human hair, preferably sodium metabisulfite and/or tourmaline. The hair straightening agents perform most of the permanent straightening through changes in the protein structure of the hair, while the surface barrier film contributes some mechanical hold, and also shields the straightened hair from ambient humidity and pollution. The invention includes compositions that may be washed out of the hair after straightening has occurred, and compositions that are intended to remain in the hair for additional or extended benefits. The invention includes methods of using a topical composition that is capable of affecting secondary, tertiary and quaternary protein structures of human hair. Testing indicates that the hair straightening is long term and there is significantly less damage to hair compared to known heat and chemical treatments.

### DESCRIPTION OF THE FIGURES

FIGS. 1a-1f show photos of surface morphology and topography of hair fibers treated with various products at 2,000× magnification.

FIGS. 2a-2f show photos of surface morphology and topography of hair fibers treated with various products at 10,000× magnification.

### DETAILED DESCRIPTION OF THE INVENTION

Throughout the specification, “topical” means applied to the surface of the hair, particularly human head hair. The word “permanent” in reference to hair straightening treatments, means that the straightened shape of the hair is maintained for at least 12 washings with a shampoo containing sodium lauryl sulfate. Preferably, if the straightened hair is exposed only to once-a-day shampooing and to ambient atmospheric conditions, the straightness of the hair is maintained for at least two weeks, more preferably, at least one month, and most preferably, at least two months. Furthermore, if treated hair is saturated (i.e. during bathing), the new shape may be lost. However, “permanent” also means that once dried, the previously saturated hair will revert to its post treatment shape, to a substantial degree. Throughout the specification, “comprising” means that a collection of objects is not necessarily limited to those recited.

### Transglutaminase/Polylysine Barrier Film

A first essential ingredient of the composition is one or more transglutaminases in combination with polylysine. As noted above, our co-pending application US2009-0126754, reported that relatively higher concentrations of TGase, when topically applied to curly hair, caused the curled hair to droop. However, while some straightening was observed, the hair was not straightened to a sufficient degree to make a hair straightening product based solely on TGase.

In compositions of the present invention, we have unexpectedly discovered that a combination of TGase and polylysine contributes to overall hair straightening. Part of this contribution may be newly acquired structure realized by the covalent bonding of protein bound lysine and protein bound glutamine. However, the topical application of TGase also appears to be capable of catalyzing the covalent bonding of polylysine supplied in a topical composition, to protein bound glutamine, near the surface of the hair. Polylysine is a natural homopolymer of the essential amino acid L-lysine, comprising approximately 25-30 L-lysine residues. It may be produced by bacterial fermentation in the genus Streptomyces. In contrast to normal peptide bonds that are linked at the alpha-carbon groups, the lysine amino acids in polylysine are linked at the epsilon amino group and the carboxyl functional group. Polylysine is a cationic polymer having a positively charged hydrophilic amino group. We have observed that if sufficient TGase and sufficient polylysine are provided in a topical hair composition, the result is a continuous film on the surface of the hair. Experiments 1 and 2 demonstrate the presence of this surface barrier film.

### Experiment 1: X-Ray Photoelectron Spectroscopy

In X-ray Photoelectron Spectroscopy (XPS), a material under investigation is bombarded with X-rays. The number and kinetic energy of electrons that escape from the top 1 to 10 nm, approximately, of the material create a spectrum, from which the elemental composition of the material may be determined. Hair samples were treated according to each of the following:
Sample 1: The base product: the base or reference product, shown as Formula 4 in Table 4, below, has no TGase, no polylysine, no calcium carbonate, and no sodium metabisulfite.
Sample 2: The base product with 0.8% polylysine added.
Sample 3: The base product with 2% transglutaminase added.
Sample 4: The base product with 2% transglutaminase, 0.8% polylysine, and 0.2% calcium carbonate added.
Sample 5: The base product with 2% transglutaminase, 0.8% polylysine, 0.2% calcium carbonate, and 2% sodium metabisulfite added.

Following treatment, the samples were washed. The washing was carried out by massaging the treated hair samples with a sodium lauryl sulfate shampoo, then rinsing with warm water. After washing, the samples were dried and left for 24 hours in a conditioning room. After this, the elemental surface compositions of each hair sample was determined. The percent elemental composition of the outermost 10 nm of the surface of each test sample is shown in table 1.

TABLE 1
Elemental surface composition of different
treated hair samples (XPS results)

| | | | % of Nitrogen | | | |
|---|---|---|---|---|---|---|
| | O | N | that is | C | S | Si |
| Sample | (1 s) | (1 s) | Ammonium-like | (1 s) | (2 p) | (2 p) |
| 1 | 22.4 | 3.5 | 12.6 | 61.9 | 0 | 12.2 |
| 2 | 17.5 | 4.1 | 9.7 | 69.4 | 0.8 | 8.2 |
| 3 | 18.6 | 3.2 | 0 | 68.4 | 0.7 | 9.1 |
| 4 | 19.7 | 5.1 | 9.7 | 66.7 | 0.8 | 7.7 |
| 5 | 16.4 | 5.4 | 19.8 | 69.7 | 1.8 | 6.7 |

Results show that nitrogen content of the outermost 10 nm of the hair surface varies from 3.2-6.4 atomic %. The portion of the nitrogen that is ammonium-like is shown in the chart. A higher ammonium-like nitrogen content is indicative of a cross-linked surface of polylysine bound to glutamine in the hair, because ammonium is a by-product of the reaction between lysine and glutamine. Sample 5 has a much higher level of ammonium-like nitrogen than the other samples (about 142% more than reference Sample 1). This is a strong indication for the reaction of hair bound glutamine with polylysine. Furthermore, the outer surface of sample 5 has least amount of silicone. Silicone is present in the base formula, but is washed away unless it is has found a binding site. That Sample 5 has the lowest amount of silicone, indicates that fewer binding sites are available to silicone, because of the formation of a cross-linked barrier surface.

Sample 4 is also interesting, having about 12 more ammonium-like nitrogen than reference Sample 1, and about 38% less silicone. These results are also indicative of the formation of cross-linked barrier surface on the hair.

The relatively large amount of sulfur in Sample 5 is from the presence of sodium metabisulfite in the product, which is still present, even after washing. The results of Samples 4 and 5 may indicate that sodium metabisulfite (present in Sample 5, but not Sample 4) is also playing a role in the formation of a cross-linked polylysine barrier surface, although it is unclear how.

### Experiment 2: Moisture Barrier Measurement

We further suspected that a surface film of polylysine would act as a moisture barrier, reducing the effects of ambient humidity on treated hair. The moisture sorption behavior of hair samples was measured with an Isothermal Gravimetric Analyzer, from Hiden Analytical. The equipment has a chamber that allows for controlled variation of humidity and temperature throughout the test. Throughout the test, a balance measures the water weight gained or lost of the fibers. Hair samples were treated according to each of the following:
Sample 1: The base product: the base or reference product, shown as Formula 4 in Table 4, below, has no TGase, no polylysine, no calcium carbonate, and no sodium metabisulfite.
Sample 5: The base product with 2% transglutaminase, 0.8% polylysine, 0.2% calcium carbonate, and 2% sodium metabisulfite added.

The base product is known to cause no damage to the hair. Prior to the start of measurement, hair samples were kept at 95% relative humidity and 22° C., for one hour, to erase the influence of the sample history. The sample was then dried at 0% relative humidity for 10 hours. Step values of relative humidity were programmed prior to the beginning of the experiment. The following sequence was used: 0%, 30%, 50%, 70%, 95%, 70%, 50%, 30%, 5%. How long a samples dwells at each level of humidity is based on a diffusion equation, and relates to the weight of the sample, but each step was a minimum time of 5 minutes and maximum time of 120 minutes. The temperature throughout was 25° C. The moisture sorption results showed that diffusion of moisture at 30-95% RH in the hair fibers of sample 5 is more hindered than that into the hair fibers of sample 1 (the base product). The degree to which the diffusion of moisture into the hair of sample 5 was hindered, compared to the reference Sample 1, is shown in table 2.

TABLE 2

| | Degree to which |
|---|---|
| | Sample 5 hindered the |
| Relative | diffusion of moisture, |
| Humidity | compared to Sample 1 |
| 30% | 2.5% |
| 50% | 8% |
| 70% | 21% |
| 95% | 20% |

This concludes that sample 5 has a moisture resistant surface, which further confirms the presence of a surface barrier film, formed by the cross-linking of formula-supplied polylysine and hair bound glutamine.

In at least two ways, this film contributes to a hair straightening composition of the present invention. First, once a polylysine film is formed on the hair via the action of TGase, the film is observed to act as a vapor and moisture barrier, that protects straightened hair from proteolytic damage, and from damage caused by environmental factors, such as ambient humidity and pollution, just to name two. Second, the continuous polylysine film, upon drying, may provide hold through the mechanical strength of the film.

We emphasize that the formation of a surface barrier film of polylysine should not be confused with the protein crosslinking that may occur between protein bound lysine and protein bound glutamine, catalyzed by TGase. To the best of our knowledge, a surface barrier film does not form from the application of TGase to the hair, in the absence of free polylysine. Nor would we expect a continuous film of this type to form on the hair from the application of TGase and non-polymerized lysine. For the surface barrier film to form, the lysine must be supplied by the topical composition, as polylysine. The moisture repelling film is covalently bonded to the hair, and does not rinse out easily, even after many washes. This barrier film is one part of the hair straightening system of the present invention, and it inhibits straightened hair from returning to a curled state.

We advise that useful concentrations of polylysine in commercially useful hair straightening compositions, are about 0.01% to about 2%; preferred is about 0.1% to about 1.5%; more preferred is about 0.5% to about 1%, based on total weight of the hair straightening composition.

The transglutaminase utilized in the present invention may be from various sources, including animal, plant and microbial sources. Available sources of transglutaminase include, but are not limited to, slime mold, alfalfa, guinea pig, and bacteria, such as Bacillus subtilis or Streptoverticillium. In terms of weight percent, the amount of transglutaminase used in a straightening product according to the present invention may vary, and will depend on the potency of the particular enzyme utilized. One preferred form of transglutaminase is available from Ajinomoto USA (Ames, IA) under the trade name Activa™TG-TI. This product is a combination of maltodextrin and powdered microbial enzyme. The TGase content of Activa™TG-TI is reported to be 0.86% to 1.35%.

### Experiment 3: Dose Response

In order to determine a dose response for the hair straightening effect of transglutaminase with 0.5% polylysine, the following procedure was performed. A grooming crème base (Table 3) adjusted to have polylysine (0.5%), calcium carbonate CaCO3 (0.5%), and urea (0.5%) was prepared with various concentrations of Activa™ TG-TI (0.5%, 1.0%, 2.0%, 4.0%, 10.0%).

TABLE 3
Grooming Crème Base

| Ingredient | Percent by weight |
|---|---|
| water | q.s. |
| petrolatum | 4.00 |
| cetearyl alcohol | 2.80 |
| polysorbate 60 | 1.20 |
| C12-15-alkyl benzoate | 1.00 |
| dipropylene glycol | 1.00 |
| sorbitan stearate | 0.75 |
| carbomer | 0.48 |
| caprylyl glycol | 0.35 |
| PEG-12 dimethicone | 0.30 |
| phenoxyethanol | 0.29 |
| safflower seed oil | 0.17 |
| sweet almond oil | 0.17 |
| castor seed oil | 0.17 |
| avocado oil | 0.17 |
| jojoba seed oil | 0.17 |
| PEG-10 soy sterol | 0.15 |
| sodium hydroxide (pH | 0.12 |
| adjuster) | |
| disodium EDTA | 0.09 |
| hexylene glycol | 0.09 |
| fragrance | 0.09 |

The compositions, which had a pH of 6.8, were applied on relatively tightly curled hair swatches, according to the following procedure: wash the swatches with shampoo and warm water; rinse well; pour sufficient amount of product in the palm of hand; apply evenly through wet hair from roots to ends; leave on hair for 45 minutes; blow dry and comb the hair straight until hair is completely dry; avoid pulling of the hair. Samples of hair so treated, were kept at ambient humidity and high humidity (about 100%) for 20 hours. Pictures were taken before and after, for comparison. By comparing before and after photos of control and test samples, it was determined that the best straightening effect was obtained with 4% Activa™ TG-TI. In this experiment, a straightening effect was not observed with 0.5% Activa™ TG-TI. An effect was observed at 1.0% and 2.0%, but was not as good as 4%. At 10% Activa™ TG-TI, the effect was about the same as at 4%.

### Experiment 4: Dwell Time

The next experiment sought to determine the length of time required for the hair straightening effect to occur, with a composition comprising the Activa™ material at 4% concentration and 0.5% polylysine. The grooming crème base of experiment 1, adjusted to have a concentration by weight of 4% Activa™ TG-TI and 0.5% polylysine, was applied to relatively tightly curled hair swatches according to the following procedure: wash the swatches with shampoo and warm water; rinse well; pour sufficient amount of product in the palm of hand; apply evenly through wet hair from roots to the ends; leave product on hair for 15, 20, 25, 30, or 45 minutes, according to sample designation; blow dry and comb the hair straight until hair is completely dry; avoid pulling of the hair. Samples of hair so treated, were kept at ambient humidity and high humidity (about 100%) for 20 hours. Pictures were taken before and after, for comparison. By comparing before and after photos of control and test samples, it was determined that a straightening effect was present in all test samples, with a maximum straightening effect achieved by 30 minutes. No additional straightening was observed in samples at 45 minutes.

### Concentration of TGase

The concentration of TGase used in a composition according to the present invention depends on the potency of the material, and the intended application. In experiment 3 above, the hair samples were tightly curled, and little or no results was seen at the 0.5% level of Activa™ TG-TI. However, we fully expect visible straightening results with as little as 0.2% of Activa™ TG-TI, when the hair being treated is less tightly curled, say mildly wavy, for example. Also, if less polylysine is used, then understandably, less catalyst (TGase) may be needed. With that in mind, we now present guidelines for concentration of Activa™ TG-TI. With these guidelines, it is well within the skill in the art to determine the optimum concentration of other transglutaminase products of different potencies. For example, another useful TGase product is a guinea pig liver transglutaminase, available from Sigma Chemical Company, having from 1.5-3.0 units of activity per mg protein.

As a guideline, for a hair straightening effect, a useful concentration of the Activa™ TG-TI material in commercially useful hair straightening compositions, is about 0.2% to about 10% (about 0.0017% to about 0.135% of transglutaminase) by weight of the total composition. A preferred concentration is between about 1.0% to about 5.0% (about 0.009% to about 0.068% transglutaminase) by weight of the total composition, particularly about 2% to about 4% (about 0.017% to about 0.054% of transglutaminase) by weight of the total composition. About 2% of Activa™ TG-TI (about 0.017% to about 0.054% of transglutaminase) is most preferred, because we have noticed that a visible dust may accumulate on the hair.

In commonly owned application US2009-0126754, it was reported that concentrations of Activa™ TG-TI below 2% promote curling of hair, when topically applied. In that case, TGase was not used in combination with polylysine. In the present case, we have noted that concentrations of Activa™ TG-TI below 2% are useful to promote straightening of hair, when applied topically, in combination with polylysine, which was not disclosed in the ‘754 application. Thus, concentrations of the Activa™ material as low as about 0.2% are also useful in the present invention, which is wholly unexpected based on US2009-0126754. Nevertheless, it may be preferable to use at least about 2% Activa™ TG-TI, if it is expected that there will be a competing curling effect from using lower concentrations.

Furthermore, as a guideline, given a composition having a 4% by weight concentration of the Activa™ material, the composition should be allowed to dwell on the hair for about 15 to about 45 minutes. A preferred time to dwell is between 25 and 45 minutes, particularly about 30 minutes. Given these guidelines, it is well within the skill in the art to determine the optimum dwell time of a composition comprising a transglutaminase whose potency is different from that of Activa™ TG-TI. However, the dwell time for effective straightening will be on the order of one hour or less and this is dramatically less than a typical known commercial straightening product, which requires 48-72 hours of dwell time, before the user can wash it out. This is a very significant advantage of the present invention.

Generally, TGase is calcium dependent, meaning the crosslinking between glutamine and lysine requires the presence of calcium. While calcium carbonate (CaCO₃) is present in hair, it is preferred if calcium is also provided in the base formula. For example, calcium in the formula may be added as CaCO₃. Typically, a useful amount of calcium carbonate might be about 0.05% to about 0.50%, for example 0.1% to 0.2%.

A second essential ingredient of the invention is one or more additional hair straightening actives, other than TGase.

### Hair Reducing Agents

Embodiments of the present invention include one or more agents that are effective to reduce cystine bonds in hair, when applied topically, in a composition disclosed herein. Of interest are sulfur containing agents, such as sulfites, thioglycolates, and cysteine; particularly bisulfites, more particularly, sodium metabisulfite.

Sulfites are hair straightening agents that straighten hair by reducing disulfide bonds (S—S) to thiosulfate bonds (—S—SO₃), also known as a Bunte salts. It has been reported in the literature that the formation of Bunte salts in hair treatment can be reversed with water rinsing, to rebuild the cystine bonds, but only relatively slowly. The rate of reversal is increased at pH of about 8 or higher. One particularly useful sulfite in some embodiments of the present invention is sodium metabisulfite, Na₂S₂O₅. As we will show, relatively less damage is done to the hair by compositions according to the invention that comprise sodium metabisulfite, while still providing effective straightening.

Thioglycolates, such as ammonium thioglycolate (a.k.a. Perm salt), and cysteine also reduce cystine bonds in hair, when applied topically, and may be used in the present invention. In solution, free ammonia swells the hair, allowing thioglycolic acid to permeate the cortex and reduces the cystine bonds, forming cysteine residues. Also, the cystine bonds may be reestablished by water rinsing or hydrogen peroxide.

The one or more agents that are effective to reduce cystine bonds in hair may typically be employed in commercially useful hair straightening compositions, at concentrations of about 0.1% to about 10% of the total weight of the composition; preferred is about 1% to about 7%; more preferred is about 2% to about 5%, by total weight of the composition.

### Activated Tourmaline

The hair shaping ability of activated tourmaline has been disclosed in commonly owned applications WO2010/096598, WO2010/096605, and WO2010/096610, herein incorporated by reference, in their entirety.

Application WO2010/096598 discloses how to make a commercially acceptable personal care composition that can supply sufficient energy for reshaping human hair via disulfide bond reorganization, while remaining reasonably priced and meeting aesthetic and regulatory requirements. It was disclosed that tourmalines heated to about 70° C. or more, emit a spectrum of light having a peak wavelength around 20 μm. Furthermore, it was shown by colorimetric disulfide bond analysis that the heated (or activated) tourmaline was effective at reducing about 6-13% of S—S bonds in hair, at acidic pH, compared to control. It was also shown, that hair straightened with activated tourmaline, according to methods disclosed therein, was not weakened to a statistically significant degree. In fact, it was shown that hair treated with activated tourmaline resulted in the formation of new secondary protein structure in the treated hair. Specifically, in the tested samples, treatment with a tourmaline-containing composition, resulted in the development of strong beta structure, strong alpha+beta structure, and strong coiled-coiled structure. Thus, it was observed that a topical composition comprising activated tourmaline is able to cleave disulfide bonds, and enhance secondary structure of hair.

In WO2010/096605, it is disclosed that hair treated by an activated tourmaline composition is effective to protect hair from thermal denaturation, as well as to increase the thermal energy required to cause protein denaturation. Tourmaline seemed to cause none of the damage to hair of the type characteristic of known heat and chemical treatments.

In WO2010/096610, it is disclosed that hair treated by an activated tourmaline composition is effective to increase the level of tightly bound water in hair. Tourmaline seemed to cause none of the damage to hair of the type characteristic of known heat and chemical treatments.

In WO2010/096598, WO2010/096605, and WO2010/096610, a useful concentration of tourmaline was disclosed to be about 1% to about 10%. In the present invention, we have observed significant hair straightening effects, when tourmaline is included in a composition that comprises TGase and polylysine. Concentrations toward the lower end, i.e. about 1% to about 4% are useful to reap some or all of the benefits of tourmaline as disclosed in WO2010/096598, WO2010/096605, and WO2010/096610. Between 1% and 2% may be preferred, and about 1% may be more preferred, to avoid a whitish residue that may result from using higher concentrations.

When using heat activated tourmaline in the present invention, it is preferable if a user applies the composition to the hair, waits a period of time during which the TGase forms the surface barrier film of polylysine, before applying heat to activate the tourmaline. This is because the heat supplied to activate the tourmaline, preferably about 70° C., is sufficient to cause break down of TGase. Therefore TGase should be allowed to work before the application of heat in excess of 30° C. Preferably, before applying heat, a user will wait about 15 to about 45 minutes. A preferred wait time is time is between 25 and 45 minutes, particularly about 30 minutes.

### Compositions

Within the guidelines, herein discussed, virtually any cosmetically acceptable or commercially viable composition, that is beneficial or benign to human hair, can serve as a base composition.

The optimum activity of transglutaminase is observed in a vehicle that has a pH from about 5 to about 9, for example a pH of about 5.0 to about 7.5. A pH of about 5.5 to 7.5 is preferred, while about 6.0 to about 7.0 is more preferred. A pH of exactly 6.0 to exactly 6.5 is most preferred. Furthermore, sodium metabisulfite reduces cysteine. Although the reaction may take place at a pH of about 3 to about 8, it is preferred if the reaction equilibrium is shifted more toward reduction of cysteine. Therefore a pH environment of about 5.5 to about 7.5 is preferred. For the same reason, a more preferred pH is about 6.0 to about 6.5.

Also, heat and/or surfactants may degrade the activity of transglutaminase. For example, heat in excess of 35-40° C. tends to degrade the stability of transglutaminase. Thus, care should be taken in handling, storage, processing, manufacture, and distribution to ensure that the TGase is not exposed to temperatures in excess of about 30° C. Furthermore, anionic surfactants, especially anionic surfactants in the presence of heat in excess of 35° C. can degrade the activity of TGase. Thus, care should be taken in formulation to select surfactants that will not significantly alter the activity of the transglutaminase under the intended conditions of use.

Compositions of the present invention must also satisfy additional criteria. For example, the compositions must be cosmetically acceptable and commercially viable. “Cosmetically acceptable” and commercially viable” or the like, usually imply that a composition is stable under typical conditions of manufacture, distribution and consumer use. By “stable”, we mean that one or more characteristics of a personal care composition do not deteriorate to an unacceptable level within some minimum period of time after manufacture. Preferably, that minimum time is six months from manufacture, more preferably one year from manufacture, and most preferably more than two years from manufacture.

Compositions of the present invention must be efficacious when used in reasonable amounts. A composition is considered effective to permanently straighten human hair, only if the amount of composition applied to the hair is what a consumer would consider reasonable. For example, if a lotion composition reshapes the hair, but a gallon of the composition is required, then this is not an effective composition according to the present invention. A person skilled in the art of personal care hair products has a very good idea of what consumers would consider reasonable. The amount of a composition of the present invention required for one treatment depends on the type and amount of hair being treated and on the desired effect. However, experience suggests that preferably, about 5 ounces, by volume, or less of a composition according to the present invention, applied to the hair, is effective to complete a treatment of a full head of hair; more preferably, about 2.0 ounces or less; most preferably, about 1.0 ounce or less. While these amounts are preferred for commercial and consumer reasons, the present invention also contemplates larger amounts, as the case may necessitate.

Furthermore, when tourmaline is used, the base composition should not absorb too much of the radiation emitted by the tourmaline, and the base composition should not interfere with activation or deactivation of the tourmaline.

Given these guidelines, compositions according to the embodiments disclosed herein, may be readily formulated into a variety of product types that are suitable for topical delivery to the hair. The composition may be a mixture, a suspension, an emulsion, a liquid, an aerosol, a gel, a cream, a lotion, a serum, or mousse, just to name a few. The composition may be in the form of a styling product, a coloring product, a conditioner or a shampoo, for example. The composition may be hydrous or substantially anhydrous. “Substantially anhydrous” means less than about 10% total water content. Methods and guidelines for formulation can be found, for example, in Harry's Cosmeticology, 8th edition, M. Reiger, Ed. 2000, the contents of which are incorporated herein by reference.

Within the guidelines discussed, a composition according to the present invention may contain any ingredients that are known to provide a benefit to the hair, any ingredients required to render a stable product, and any ingredients that render the product more cosmetically acceptable or commercially viable. For example, compositions according to the present invention may advantageously contain hair coloring agents. Hair coloring reactions of the type well known in the art, and disulfide bond cleavage as described herein, may exhibit synergistic effects.

### Hair Damage Testing

The compositions of table 4 are cosmetically acceptable and commercially viable compositions according to the present invention. Each contains 2% Activa™ TI (1% transglutaminase/99% maltodextrin), 0.8% polylysine, 0.2% CaCO₃; 2.75% sodium metabisulfite in formulae 1 and 2, 2.0% sodium metabisulfite in formula 3, and red tourmaline at 1%. Formula 1 has a pH of 5.5, Formula 2 has a pH of 5.21, and Formula 3 has a pH of 5.5.

TABLE 4

| | Percent by weight of composition | | | |
|---|---|---|---|---|
| Ingredients | Formula 1 | Formula 2 | Formula 3 | Formula 4 |
| water | q.s. | q.s. | q.s. | q.s. |
| phenyl trimethicone | 17.0 | 10.0 | 5.0 | 5.0 |
| Dimethicone/ | 9.0 | 9.0 | 9.7 | 9.7 |
| dimethiconol | | | | |
| propanediol | 7.0 | 14.0 | 36.0 | 36.0 |
| hexylene glycol | 5.1 | 5.1 | 5.1 | 5.1 |
| dimethicone | 5.0 | 5.0 | 14.0 | 14.0 |
| PEG/PPG-18/18 | 3.0 | 3.0 | 1.0 | 1.0 |
| dimethicone | | | | |
| sodium metabisulfite | 2.75 | 2.75 | 2.0 | 2.0 |
| Activa ™ TI (#) | 2.0 | 2.0 | 2.0 | |
| red tourmaline | 1.0 | 1.0 | 1.0 | |
| phenoxyethanol/ | 1.0 | 1.0 | 1.0 | 1.0 |
| caprylyl glycol/ | | | | |
| potassium sorbate/ | | | | |
| water/hexylene glycol | | | | |
| guar | 0.8 | 0.8 | — | — |
| hydroxypropyltrimonium | | | | |
| chloride | | | | |
| polylysine 25% aq. | 0.8 | 0.8 | 0.8 | |
| solution | | | | |
| citric acid | 0.3 | 0.3 | 0.3 | 0.3 |
| calcium carbonate | 0.2 | 0.2 | 0.2 | |
| bismuth oxychloride | 0.001 | 0.001 | 0.001 | 0.001 |
| sapphire powder | 0.001 | 0.001 | 0.001 | 0.001 |
| malachite | 0.001 | 0.001 | 0.001 | 0.001 |
| dimethicone/ | 0.0001 | 0.1 | 0.1 | 0.1 |
| mercaptopropyl/ | | | | |
| methicone copolymer | | | | |
| dimethicone/ | 0.0001 | 0.1 | 0.1 | 0.1 |
| mercaptopropyl/ | | | | |
| methicone copolymer/ | | | | |
| phenyl trimethicone | | | | |
| polyacrylamide/C13-14 | — | — | 3.0 | 3.0 |
| isoparaffin/laureth-7 | | | | |
| ceramide 2 | — | — | 0.1 | 0.1 |

(#) - 1% transglutaminase/99% maltodextrin

These were tested for damage inflicted on hair, and compared to the following commercially available products: Perm A (a cysteine-based product; pH=about 9); Perm B (a 2% formaldehyde product); Perm C (a 2% sodium hydroxide product; pH=about 13.4); Perm D (a 10% ammonium thioglycolate, 0.3% dithioglycolic acid product).

### Experiment 5: Hair Cystine (S—S) Bond Measurements

Untreated hair is reported to contain about 750-1,534 μmoles of disulfide bonds per gram. Cleaving of disulfide bonds by some hair straightening treatments represents a significant loss of structure, which could be characterized as damage to the hair. The degree of loss of disulfide bonds in hair, as a result of various hair straightening treatments, was quantified. A colorimetric assay was used to determine the quantity of S—S bonds, by directly measuring the amount of oxidized dithiothreitol (DTT) in hair. Dithiothreitol (C₄H₁₀O₂S₂) is a strong reducing agent which is oxidized in reaction with cystine. The reduction of the disulfide bond in cystine proceeds by two sequential thiol-disulfide exchange reactions. In the process, oxidized DTT is stoichiometrically produced. It is stable and has UV-absorption spectrum with λₘₐₓ=280 nm. Results are shown in table 5, where a smaller percent reduction in S—S bonds means less damage.

TABLE 5

| | | % reduction of |
|---|---|---|
| Products | Hair Straightening Ingredients | S—S bonds |
| Control: Untreated | — | — |
| Hair | | |
| Formula 1 | 2% Transglutaminase | 12% |
| | 0.8% Polylysine | |
| | 0.2% CaCO₃ | |
| | 1% Tourmaline | |
| | 2.75% Na₂S₂O₅ | |
| | pH = 5.50 | |
| Formula 2 | 2% Transglutaminase | 12% |
| | 0.8% Polylysine | |
| | 0.2% CaCO₃ | |
| | 1% Tourmaline | |
| | 2.75% Na₂S₂O₅ | |
| | pH = 5.21 | |
| Perm A | Cysteine, pH = 9 | 24% |
| Perm B | 2% Formaldehyde | 25% |
| Perm C | 2% Sodium Hydroxide, | 27% |
| | pH = 13.4 | |
| Perm D | 10% ammonium thioglycolate, | 25% |
| | dithioglycolic acid 0.3% | |

### CONCLUSION

The results in Table 5 show that the two formula according to the present invention cause less than half the damage of several commercially available products.

### Experiment 6: Contact Angle Measurement

Untreated hair fibers are naturally hydrophobic, so that the surface of the fiber is water resistant. This is due to the presence of 18-MEA. However, when hair is damaged, it loses water resistance, even becoming hydrophilic. The wetting properties of hair fibers can be quantified by measuring the contact angle that water makes with the surface of the fibers. When the water contact angle is greater than or equal to 90° (the water pulls away from the surface), the surface is considered hydrophobic. When the water contact angle is less than 90° (the water spreads out on the surface), the surface is considered hydrophilic.

Individual hair fibers were mounted in a fiber holder. A 50 μl syringe and glass dosing needle were used to deposit distilled water droplets (about 10 nanoliter) onto the hair surface. The process of wetting the fibers was video recorded, at a rate of 5 images per second, for 60 seconds. The contact angles were automatically calculated for each image using manual fitting. The average of three contact angle measurements is reported in Table 6, for hair fibers treated by various straightening methods.

TABLE 6

| | | | Change in |
|---|---|---|---|
| | Hair Straightening | Contact | Contact |
| Products | Ingredients | Angle | Angle |
| Control: Untreated | None | 105° ± 2° | — |
| Hair | | | |
| Formula 1 | 2% Transglutaminase | 90° ± 2° | −14% |
| | 0.8% Polylysine | | |
| | 0.2% CaCO₃ | | |
| | 1% Tourmaline | | |
| | 2.75% Na₂S₂O₅ | | |
| | pH = 5.50 | | |
| Formula 2 | 2% Transglutaminase | 91° ± 4° | −13% |
| | 0.8% Polylysine | | |
| | 0.2% CaCO₃ | | |
| | 1% Tourmaline | | |
| | 2.75% Na₂S₂O₅ | | |
| | pH = 5.21 | | |
| Perm A | Cysteine, pH = 9 | 64° ± 3° | −39% |
| Perm B | 2% Formaldehyde | 60° ± 2° | −43% |
| Perm C | 2% Sodium Hydroxide, | 51° ± 40° | −51% |
| | pH = 13.4 | | |
| Perm D | 10% ammonium | 62° ± 3° | −41% |
| | thioglycolate, 0.3% | | |
| | dithioglycolic acid | | |

### CONCLUSION

The results in table 6 show that the hair fibers treated with two formula according to the present invention retain a natural hydrophobic character. In contrast, as a result of damage incurred, all of the hair fibers treated with commercial products lose their natural hydrophobic character, and display hydrophilic character. Hair samples treated with compositions according to the present invention show only about one third as much damage as the nearest commercial product, as measured by water contact angle.

### Experiment 7: Scanning Electron Microscopy

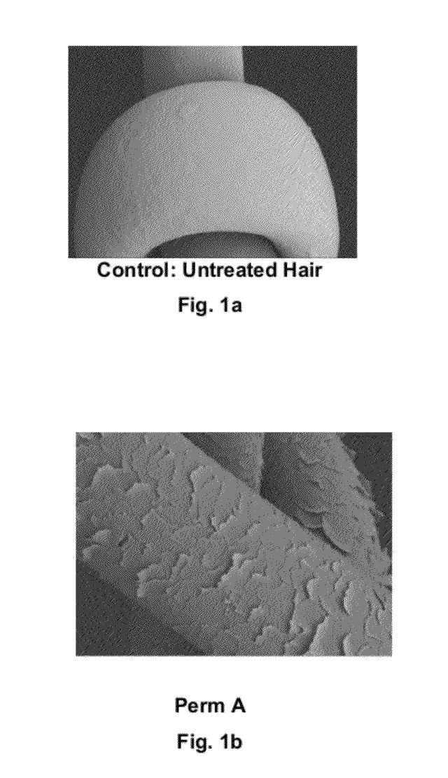

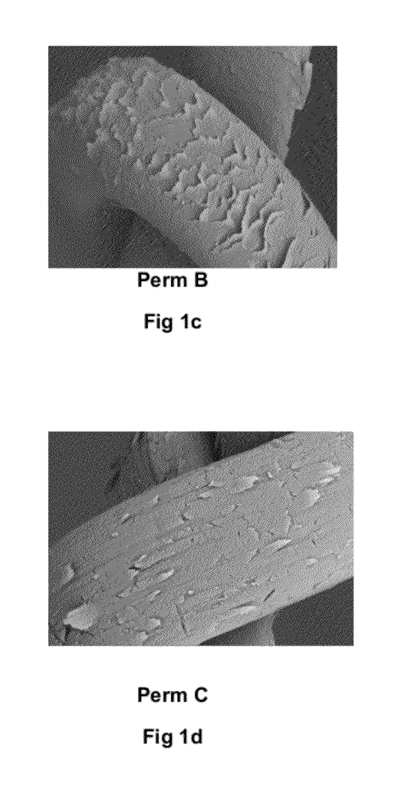

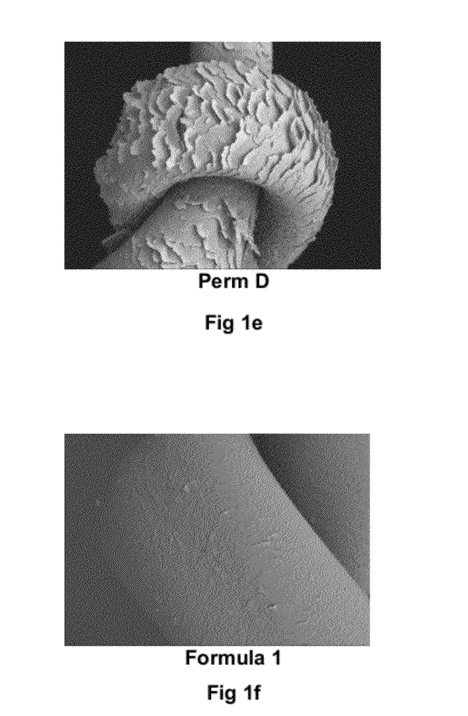

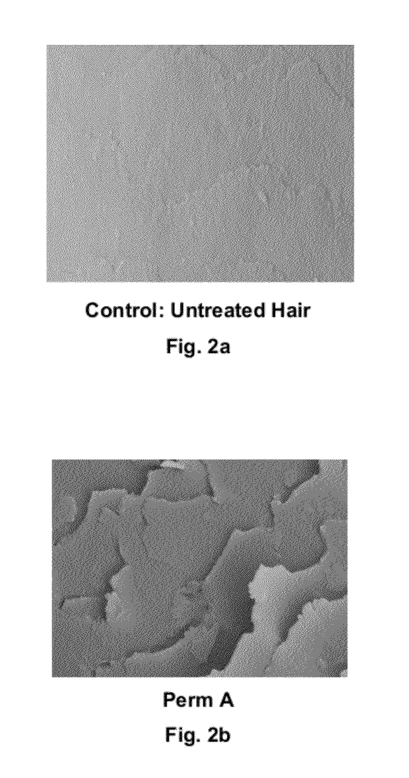

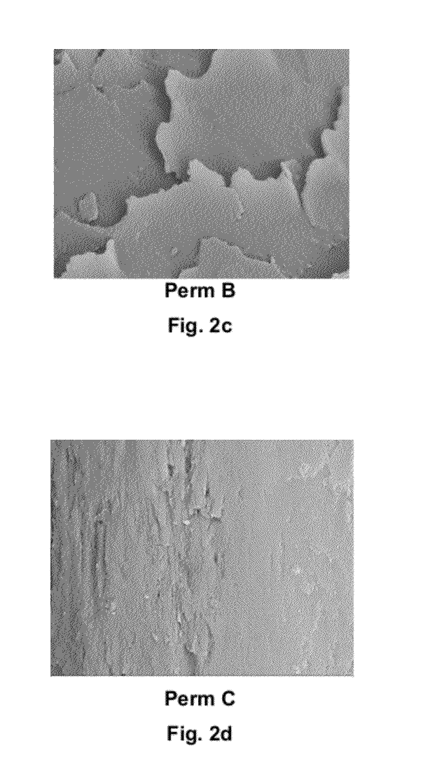

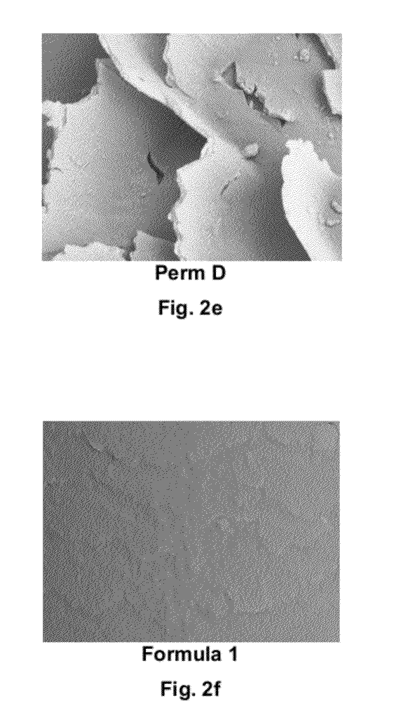

Scanning Electron Microscopy was used to analyze the surface morphology and topography of hair fibers treated with the same products as above. The results are shown in FIGS. 1 and 2. FIG. 1 shows hair fibers at 2000× magnification, and FIG. 2 shows the same fibers at 10,000× magnification. In summary, the surface of untreated hair (FIGS. 1a, 2a) is undamaged, meaning the surface is smooth and the cuticle is in tact. In contrast, the surfaces of the fibers treated with Perm C (1d, 2d), Perm A (1b, 2b), Perm B (1c, 2c), and Perm D (1e, 2e) are irregular, and show signs of lifting cuticles. In fact, in the Perm C sample, the cuticle has been removed almost completely. In obvious contrast, the hair fiber surface treated with Formula 1, according to the present invention, shows very minimal damage, i.e. the surface is smooth and has uniform cuticle surface (FIGS. 1f, 2f), comparable in appearance to the untreated hair sample. This photographic evidence is the strongest possible evidence of the performance advantages of compositions of the present invention, vis-á-vis hair damage.

### Experiment 8: Tensile Testing

An Instron Testing machine was used to measure the mechanical properties of hair such as: resistance to stretching, friction, tensile strength and elasticity. Tensile strength results are in terms of Young's modulus, i.e. a higher Young's modulus means greater. strength. Each test sample of hair comprised about 50 fibres, each 50 mm long, with roots and tips removed. The samples were conditioned for 24 hours at 55% relative humidity, and 22° C. The cross sectional area of the fibers was measured with the fiber dimensional analysis system at several points along the fiber length. The Instron machine was used to record a stress-strain curve for each hair sample. From this, the following may be readily determined: Young's modulus, yield plateau, the breaking point, and work performed. This procedure was carried out on hair samples like those of Experiment 2, above.
Sample 1: The base product: the base or reference product, shown as Formula 4 in Table 4, above, has no TGase, no polylysine, no calcium carbonate, and no sodium metabisulfite.
Sample 5: The base product with 2% transglutaminase, 0.8% polylysine, 0.2% calcium carbonate, and 2% sodium metabisulfite added.

The product applied to hair Sample 1 is a reference product that is known to cause no damage to hair. Young's modulus was the same, about 1.3 GPa, for Samples 1 and 5. Likewise, the results for yield plateau, breaking point, and work performed were very similar for both samples. This indicates that a composition according to the present invention produced no more macroscopic mechanical damage to the hair, than an inactive base formula.

In summary, compositions according to the present invention are decidedly less damaging to hair than several commercially available products tested, which employ cysteine, formaldehyde, sodium hydroxide, or ammonium thioglycolate and dithioglycolic acid, while still being effective to straighten the hair.

### Methods of Use

A composition according to the present invention is applied to swatch of non-straightened hair. The swatch of hair may be wet (preferred) or dry (less preferred) when the composition is applied. Preferably, the composition is applied evenly from the roots to the ends. For example, at least 50% of the hair, from the root to the end is coated with the composition, more preferably at least 75%, most preferably at least 95%.

After the composition is applied to the hair, the composition should be allowed to dwell on the hair for a minimum dwell time, before further treatment. For example, it is preferable if the dwell time is at least 10-20 minutes, more preferably at least 20-30 minutes, even more preferably at least 35 minutes, and most preferably 30-35 minutes. During the dwell time, it is especially important that the composition on the treated hair is not exposed to temperatures in excess of 45° C., preferably not in excess of about 40° C., and more preferably not in excess of about 35° C.

After the dwell time, the hair should be combed straight while blow drying with a hot air blow dryer. Excessive pulling of the hair is not necessary and should be avoided. Preferably, as a result of blow drying, the hair achieves a temperature of at least 50° C., more preferably at least 60°, and most preferably at least 70° C., to activate the tourmaline.

What is claimed is:

1. A hair straightening composition comprising:
a cosmetically acceptable base;
0.01% -2.0% of polylysine by total weight of the composition; and
0.0017% -0.135% of transglutaminase by total weight of the composition;
the overall pH of the composition being 5.0 to 7.5.

2. The composition of claim 1 wherein the overall pH is 6.0 to 6.5.

3. The composition of claim 1 further comprising calcium.

4. The composition of claim 3 wherein the calcium is in the form of calcium carbonate.

5. The composition of claim 4 further comprising one or more agents that are effective to reduce cystine bonds in hair.

6. The composition of claim 5 comprising one or more sulfites.

7. The composition of claim 6 comprising one or more bisulfites.

8. The composition of claim 7 comprising sodium metabisulfite, Na₂S₂O₅, at a concentration of 0.1% to 10%, by total weight of the composition.

9. The composition of claim 5 comprising one or more tourmalines at a concentration of 1.0% to 10%, by total weight of the composition.

10. The composition of claim 8 comprising one or more tourmalines at a concentration of 1.0% to 10%, by total weight of the composition.

11. The composition of claim 2 wherein the concentration of polylysine is about 0.5% to about 1%.

12. The composition of claim 10 wherein the concentration of polylysine is about 0.5% to about 1%.

13. A method of straightening hair, comprising the steps of:
providing a composition according to claim 1;
applying 5 ounces or less of the composition to non-straightened hair;
allowing the composition to dwell on the hair for a dwell time of at least 20-30 minutes; and
during the dwell time, not exposing the composition on the hair to temperatures in excess of 45° C.

14. A method of straightening hair, comprising the steps of:
providing a composition according to claim 8;
applying 5 ounces or less of the composition to non-straightened hair;
allowing the composition to dwell on the hair for a dwell time of at least 20-30 minutes; and
during the dwell time, not exposing the composition on the hair to temperatures in excess of 45° C.

15. A method of straightening hair, comprising the steps of:
providing a composition according to claim 9;
applying 5 ounces or less of the composition to non-straightened hair;
allowing the composition to dwell on the hair for a dwell time of at least 20-30 minutes;
during the dwell time, not exposing the composition on the hair to temperatures in excess of 45° C.; and
after the dwell time, activating the tourmaline by heating the composition on the hair to at least 50° C.

16. A method of straightening hair, comprising the steps of:
providing a composition according to claim 10;
applying 5 ounces or less of the composition to non-straightened hair;
allowing the composition to dwell on the hair for a dwell time of at least 20-30 minutes;
during the dwell time, not exposing the composition on the hair to temperatures in excess of 45° C.; and
after the dwell time, activating the tourmaline by heating the composition on the hair to at least 50° C.
